(19) United States
(12) Patent Application Publication
(10) Pub. No.: US 2018/0042972 A1
(43) Pub. Date: Feb. 15, 2018

(54) PROBIOTIC FORMULATIONS

(71) Applicant: SmartyPants Inc., Marina del Rey, CA (US)

(72) Inventors: Courtney Nichols GOULD, Marina del Rey, CA (US); Michael CLEARY, Vancouver, WA (US)

(21) Appl. No.: 15/400,894

(22) Filed: Jan. 6, 2017

Related U.S. Application Data
(63) Continuation of application No. 15/233,825, filed on Aug. 10, 2016, now abandoned.

(51) Int. Cl. A61K 35/742; A61K 47/36; A23L 33/135; A61K 9/50

(52) U.S. Cl. CPC A61K 35/742; A61K 9/5063; A61K 47/36; A23L 33/135; A61K 2035/11

(57) ABSTRACT

Described herein are formulations for providing one or more active probiotics to an individual. Compositions are described herein comprising a base substance, one or more prebiotics, and one or more probiotics. Such compositions are beneficial in that they, for example, promote GI health and enhancing immunity.

CROSS-REFERENCE

This application is a continuation/continuation-in-part application of Ser. No. 15/233,825, filed Aug. 10, 2016,which is incorporated herein by reference in its entirety and to which application we claim priority under 35 USC § 120.

### BACKGROUND

Probiotics are generally grouped within the category of foods and nutritional supplements such as vitamins and minerals. Probiotics typically comprise micro-organisms that are able to provide beneficial effects when ingested.

For example, certain probiotics have been found to stimulate the immune system, and certain probiotics have been found to aid in digestion. In another example, specific probiotics may be provided to patients to provide the health benefits associated with the specific probiotic to the patient.

Prebiotics are a general class of nutrient that can enhance the activity of the microbiome, and specifically probiotics thus providing an indirect but nonetheless beneficial effect on health. Furthermore, specific prebiotics may provide health benefits in addition to microbiome or probiotic activity.

### SUMMARY

Described herein are formulations for providing one or more active probiotics to an individual. Formulations described herein are configured for oral ingestion by a human or animal in order to provide an active probiotic is capable of providing systemic and/or gastro-intestinal (“GI”) benefits to the human or animal who has ingested said formulation. In the typical embodiment described herein, an active probiotic comprises a living micro-organism. The living micro-organisms described herein exert their beneficial effects on the individual who ingested the formulations described herein through an initial colonization of the GI tract of the individual.

In some embodiments of the formulations described herein, the probiotic is delivered in a confection. Confections described herein are prepared to be satisfying to the individual who ingests them by providing, for example, pleasing texture and taste.

Confections described herein are prepared so that loss of active probiotics is minimized. In some embodiments, a confection comprises a gel such as, for example, a gummy. In these embodiments, the probiotic along with other components of the formulation are incorporated within the gel and the gel thereby encloses the various ingredients of the formulation including the probiotics.

In some embodiments of the formulations described herein, a probiotic composition comprises a prebiotic. A prebiotic generally comprises an ingredient that promotes or enhances the benefit provided by the probiotic. A prebiotic may provide additional benefits as well including, for example, immune enhancing or aiding digestion.

In some embodiments, a composition as described herein comprises a plurality of micro-organisms. For example, in some embodiments, a composition comprises a first micro-organism and a second micro-organism. In some of these embodiments, the first and the second micro-organism are each selected from a different genus of bacteria. In some of these embodiments, the first and the second micro-organism are each selected from a different species of bacteria. In some embodiments, the first and the second micro-organism are each selected from a different strain of bacteria. In some embodiments, the composition comprises at least one spore-forming bacterial species.

Described herein are methods of manufacture of probiotic compositions. In some embodiments, spore-forming bacteria in a dormant state (i.e. as spores) are mixed together with one or more ingredients. In some embodiments, a gel is formed by heating the components of the gel and then it is allowed to cool, following which bacterial spores are added. In some embodiments, a prebiotic is mixed together with the cooled gel and the bacterial spores. In some embodiments, additional ingredients are added to provide texture and taste including, for example, an acid such as citric acid.

Described herein is a composition comprising: a hydrocolloid; beta-glucan; a first spore forming bacteria; and a second spore forming bacteria; wherein the composition comprises a mixture of the hydrocolloid, the beta-glucan, the first spore forming bacteria, and the second spore forming bacteria. In some embodiments, the hydrocolloid comprises at least one of a gum, a gelatin, a starch, a gel, and a pectin. In some embodiments, the composition comprises a wax coating that encapsulates the composition. In some embodiments, the hydrocolloid, comprises 85% by weight or more of carbohydrates. In some embodiments, the hydrocolloid comprises 1% by weight or more of an acid. In some embodiments, the beta-glucan is derived from a grain or a fungus. In some embodiments, the composition comprises from about 35 mg to about 200 mg of beta-glucan. In some embodiments, the first spore forming bacteria and the second spore forming bacteria are each selected from a group consisting of Bacillus subtilis, Bacillus clausii, Bacillus cereus, Bacillus coagulans and Bacillus licheniformis. In some embodiments, the first spore forming bacteria comprises about 1.5 billion to about 2.5 billion colony forming units of Bacillus subtilis and the second spore forming bacteria comprises about 0.5 billion to about 1 billion colony forming units of Bacillus coagulans. In some embodiments, the composition comprises a homogenous mixture.

Described herein is a method of treating an individual, comprising: providing the individual with a composition comprising a mixture of a hydrocolloid, a beta-glucan, a first spore forming bacteria, and a second spore forming bacteria. In some embodiments, the hydrocolloid comprises at least one of a gum, gelatin, starch and pectin. In some embodiments, the composition comprises a wax coating that encapsulates the composition. In some embodiments, the hydrocolloid comprises 85% by weight or more of carbohydrates. In some embodiments, the hydrocolloid comprises 1% by weight or more of an acid. In some embodiments, the beta-glucan is derived from a grain or a fungus. In some embodiments, the composition comprises from about 35 mg to about 200 mg of beta-glucan. In some embodiments, the first spore forming bacteria and the second spore forming bacteria are each selected from a group consisting of Bacillus subtilis, Bacillus clausii, Bacillus cereus, Bacillus coagulans and Bacillus licheniformis. In some embodiments, the first spore forming bacteria comprises about 1.5 billion to about 2.5 billion colony forming units of Bacillus subtilis and the second spore forming bacteria comprises about 0.5 billion to about 1 billion colony forming units of Bacillus coagulans. In some embodiments, the composition comprises a homogenous mixture.

Also described herein is a composition comprising: a gel; a first spore forming bacteria; and a second spore forming bacteria. In some embodiments, the gel comprises a hydrocolloid. In some embodiments, the hydrocolloid comprises at least one of gum, gelatin, cornstarch and pectin. In some embodiments, the first spore forming bacteria comprises any of Bacillus subtilis, Bacillus clausii, Bacillus cereus, Bacillus coagulans and Bacillus licheniformis. In some embodiments, the gel comprises 85% by weight or more of carbohydrates. In some embodiments, the gel comprises 1% by weight or more of an acid. In some embodiments, the second spore forming bacteria comprises any of Bacillus subtilis, Bacillus clausii, Bacillus cereus, Bacillus coagulans and Bacillus licheniformis. In some embodiments, the composition comprises about 2.5 billion colony forming units of the first spore forming bacteria and about 1 billion colony forming units of the second spore forming bacteria. In some embodiments, the composition comprises about 1.5 billion colony forming units of the first spore forming bacteria and about 0.5 billion colony forming units of the second spore forming bacteria. In some embodiments, the composition has a wax coating. In some embodiments, the composition comprises a homogenous mixture.

Described herein is a composition comprising: a prebiotic; a first spore forming bacteria; and a second spore forming bacteria. In some embodiments, the prebiotic comprises at least one of fructooligosaccharide, galactooligosaccharide, lactulose, beta-glucan, inulin, and digestion resistant starch. In some embodiments, the first spore forming bacteria comprises any of Bacillus subtilis, Bacillus clausii, Bacillus cereus, Bacillus coagulans and Bacillus licheniformis. In some embodiments, the second spore forming bacteria comprises any of Bacillus subtilis, Bacillus clausii, Bacillus cereus, Bacillus coagulans and Bacillus licheniformis.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

Formulation

Described herein are formulations for the delivery of a probiotic. The formulations described herein comprise edible compositions that deliver one or more active agents. The compositions described herein are configured to release one or more active ingredients into the GI system of an individual who ingests the composition, wherein the active ingredients are selected to exert either local (i.e. local to the GI tract) or systemic effects or both on the individual. As used herein an individual may comprise a human or animal.

The compositions described herein include a base substance that carries or contains the active ingredients of the formulation. A base substance may comprise a solid, liquid, or semi-solid edible substance depending on the particular embodiment of the composition. For example, some embodiments of the compositions as described herein comprise a confection such as, for example, a gummy containing one or more active ingredients. In such embodiments, the gummy comprises a base substance that contains the active ingredients of the composition.

In general, a base substance as described herein is configured to provide a number of qualities to the composition including, for example, enhanced palatability of the active ingredients by providing, for example, a satisfying taste or texture to the composition. Additionally, in some embodiments, a base substance as described herein provides a delivery vehicle for an active ingredient by, for example, encapsulating a micro-organism in a protective environment (where the micro-organism comprises the active ingredient). For example, a base substance as described herein, in some embodiments, encapsulates a micro-organism in an oxygen free or essentially oxygen free environment.

In some embodiments, the composition of the base substance itself is configured to control the mechanism of release or timing of release of the active ingredient. For example, a base substance that is resistant to digestion by stomach acid will release active ingredients further along the GI tract than a base substance that is digested by stomach acid. In this way, targeted delivery of specific probiotics to specific targets in the GI tract is achievable. For example, probiotics for reducing or preventing H. pylori colonization of the stomach is best delivered via a base substance that is digested quickly in the upper GI tract, while, for example, probiotics for the treatment of Inflammatory Bowel Syndrome are best delivered to the colon via a base substance that takes a relatively longer time to digest. The mechanism of digestion of the base unit is further controlled in some embodiments by the addition of a coating of the entire composition.

In some of the embodiments of the compositions described herein, a base substance comprises a colloid or a hydrocolloid. In some of these embodiments, a hydrocolloid is a gum. In some of these embodiments, a hydrocolloid is a gelatin. In some of these embodiments a hydrocolloid is a starch. In some of these embodiments, a hydrocolloid is a pectin. In some of these embodiments, a hydrocolloid is a gel. In general, a hydrocolloid is formed by mixing a powdered or dehydrated base substance with water. In some embodiments, the mixture of water and powdered base substance is heated to form the hydrocolloid. In some embodiments, the mixture of water and powdered base substance is heated under pressure to form the hydrocolloid. In general, the concentration of the powdered substance relative to the water in the mixture will determine the consistency of the hydrocolloid. As such, in some embodiments, to obtain a more solid consistency, water is intentionally evaporated from the hydrocolloid, thus increasing the relative concentration of the powdered base substance. Similarly, water may be added to a hydrocolloid to decrease the relative concentration of the powdered base substance and thus cause the hydrocolloid to become relatively more liquid or semi-solid. In some embodiments wherein the compositions as described herein comprise a hydrocolloid as the base substances, the base substance is poured into a mold and formed into a gummy.

In some embodiments of the compositions described herein, the base unit is a soft chew having, for example, a fondant-like texture and consistency. Exemplary components of a soft chew base unit include one or more of a sweetener in the form of, for example, a syrup, glycerin, and gelatin.

In some embodiments of the compositions described herein, additives provide or enhance flavoring, texture, and/or coloring. Exemplary flavorants include salts, acids, and sweeteners. Acids such as, for example, citric acid, in addition to providing flavor, provide texture by promoting the gelling of hydrocolloids in embodiments that include hydrocolloids. In some embodiments, citric acid is further combined with one or more of lactic acid, malic acid and ascorbic acid. In some embodiments, the base substance in the composition comprises 5% by weight of an acid. In some embodiments, the base substance in the composition comprises 4% by weight of an acid. In some embodiments, the base substance in the composition comprises 3% by weight of an acid. In some embodiments, the base substance in the composition comprises 2% by weight of an acid. In some embodiments, the base substance in the composition comprises 1% by weight of an acid. In some embodiments, the base substance in the composition comprises no acid. Sweeteners such as, for example, sucrose, fructose, glucose or other sugars, invert sugar, or syrup. In some embodiments, a sweetener comprises an artificial sweetener. In some embodiments, sweetener is incorporated into the base substance in the composition and/or sweetener is layered or coated onto one or more surfaces of the hydrocolloid. In some embodiments, the base substance in the composition comprises about 85%-95% carbohydrates by weight. In some embodiments, the base substance in the composition comprises about 95% carbohydrates by weight. In some embodiments, the base substance in the composition comprises about 90% carbohydrates by weight. In some embodiments, the base substance in the composition comprises about 85% carbohydrates by weight. In some embodiments, the base substance in the composition comprises about 80% carbohydrates by weight. In some embodiments, the base substance in the composition comprises about 75% carbohydrates by weight. In some embodiments, the base substance in the composition comprises about 70% carbohydrates by weight. In some embodiments, the base substance in the composition comprises about 65% carbohydrates by weight. In some embodiments, the base substance in the composition comprises about 60% carbohydrates by weight. In some embodiments, the base substance in the composition comprises about 55% carbohydrates by weight. In some embodiments, the base substance in the composition comprises about 50% carbohydrates by weight. In some embodiments, the base substance in the composition comprises about 45% carbohydrates by weight. In some embodiments, the base substance in the composition comprises 40% carbohydrates by weight. In some embodiments, the base substance in the composition comprises about 35% carbohydrates by weight. In some embodiments, the base substance in the composition comprises about 30% carbohydrates by weight. In some embodiments, the base substance in the composition comprises about 25% carbohydrates by weight. In some embodiments, the base substance in the composition comprises about 20% carbohydrates by weight. In some embodiments, the base substance in the composition comprises about 15% carbohydrates by weight. In some embodiments, the base substance in the composition comprises about 10% carbohydrates by weight. In some embodiments, the base substance in the composition comprises about 5% carbohydrates by weight. In some embodiments, the base substance in the composition comprises no carbohydrates.

In a preferred embodiment of the compositions described herein, the composition includes one or more active ingredients.

Exemplary active ingredients suitable for use in the compositions described herein include prebiotics, probiotics, vitamins, minerals, and other dietary supplement nutrients (e.g. oils, amino acids).

Exemplary prebiotics suitable for use in the compositions described herein include fructooligosaccharide, galactooligosaccharide, lactulose, beta-glucan, inulin, pectin and digestion resistant starch. In some embodiments of the compositions described herein, the compositions comprise one or more prebiotics. In some embodiments of the compositions described herein, the compositions comprise one prebiotic. In some embodiments of the compositions described herein, the compositions comprise two prebiotics. In some embodiments of the compositions described herein, the compositions comprise three prebiotics. In some embodiments of the compositions described herein, the compositions comprise four prebiotics. In some embodiments of the compositions described herein, the compositions comprise five prebiotics. In some embodiments of the compositions described herein, the compositions comprise six prebiotics. In some embodiments of the compositions described herein, the compositions comprise seven prebiotics. In some embodiments of the compositions described herein, the compositions comprise eight prebiotics. In some embodiments of the compositions described herein, the compositions comprise nine prebiotics. In some embodiments of the compositions described herein, the compositions comprise 10 prebiotics.

Exemplary probiotics suitable for use in the compositions described herein include strains from the following genera: Lactobacillus, Bifidobacterium, Saccharomyces, Enterococcus, Streptococcus, Pediococcus, Leuconostoc, Bacillus, Escherichia coli. Microorganisms from these genera in particular have been demonstrated to provide health benefits when ingested. See, for example, S. Fijan. Microorganisms with Claimed Probiotic Properties: An Overview of Recent Literature. Int J Environ Res Public Health. 2014 May; 11(5): 4745-4767. Non-limiting examples of specific micro-organisms suitable for use in the compositions described herein include Lactobacillus acidophilus, L. casei, L. paracasei, L. rhamnosus, L. delbrueckii subsp. bulgaricus, L. brevis, L. johnsonii, L. plantarum, L. fermentum, Bifidobacterium infantis, B. adolescentis, B. animalis subsp animalis, B. animalis subsp lactis, B. bifidum, B. longum, B. breve, B. infantis, Saccharomyces bayanus, S. cervisiae, S. boulardii. Lactococcus lactis subsp. Lactis, Streptococcus pyogenes, Streptococcus pneumoniae, S. thermophiles, vancomycin-resistant Enterococcus faecium, B. subtilis, B. coagulans, B. subtilis, B. cereus, B. indicus, B. licheniformis, and Escherichia coli. In some embodiments of the compositions described herein, the compositions comprise one or more types (e.g. one or more species or one or more genera) of probiotics. In some embodiments of the compositions described herein, the compositions comprise one probiotic. In some embodiments of the compositions described herein, the compositions comprise two types of probiotics. In some embodiments of the compositions described herein, the compositions comprise three types of probiotics. In some embodiments of the compositions described herein, the compositions comprise four types of probiotics. In some embodiments of the compositions described herein, the compositions comprise five types of probiotics. In some embodiments of the compositions described herein, the compositions comprise six types of probiotics. In some embodiments of the compositions described herein, the compositions comprise seven probiotics. In some embodiments of the compositions described herein, the compositions comprise eight types of probiotics. In some embodiments of the compositions described herein, the compositions comprise nine types of probiotics. In some embodiments of the compositions described herein, the compositions comprise 10 types of probiotics. In some embodiments of the compositions described herein, the compositions comprise one or more spore-forming microbial types. In some embodiments, the compositions comprise 1 spore-forming microbial types. In some embodiments, the compositions comprise 2 spore-forming microbial types. In some embodiments, the compositions comprise 3 spore-forming microbial types. In some embodiments, the compositions comprise 4 spore-forming microbial types. In some embodiments, the compositions comprise 5 spore-forming microbial types. In some embodiments, the compositions comprise 6 spore-forming microbial types. In some embodiments, the compositions comprise 7 spore-forming microbial types. In some embodiments, the compositions comprise 8 spore-forming microbial types. In some embodiments, the compositions comprise 9 spore-forming microbial types. In some embodiments, the compositions comprise 10 spore-forming microbial types.

Exemplary vitamins suitable for use with the compositions described herein include Vitamin A, Vitamin B1, Vitamin B2, Vitamin B3Niacin, Vitamin B5Pantothenic Acid, Vitamin B6Pyridoxine, Vitamin B7Biotin, Vitamin B9Folate, Vitamin B12, Vitamin C, Vitamin D, Vitamin E, Vitamin K, and Choline. In some embodiments of the compositions described herein, the compositions comprise one or more vitamins. In some embodiments of the compositions described herein, the compositions comprise one vitamin. In some embodiments of the compositions described herein, the compositions comprise two vitamins. In some embodiments of the compositions described herein, the compositions comprise three vitamins. In some embodiments of the compositions described herein, the compositions comprise four vitamins. In some embodiments of the compositions described herein, the compositions comprise five vitamins. In some embodiments of the compositions described herein, the compositions comprise six vitamins. In some embodiments of the compositions described herein, the compositions comprise seven vitamins.

In some embodiments of the compositions described herein, the compositions comprise eight vitamins. In some embodiments of the compositions described herein, the compositions comprise nine vitamins. In some embodiments of the compositions described herein, the compositions comprise ten vitamins. In some embodiments of the compositions described herein, the compositions comprise eleven vitamins. In some embodiments of the compositions described herein, the compositions comprise twelve vitamins. In some embodiments of the compositions described herein, the compositions comprise thirteen vitamins. In some embodiments of the compositions described herein, the compositions comprise fourteen vitamins.

Exemplary minerals suitable for use with the compositions described herein include Potassium, Chloride, Sodium, Calcium, Phosphorus, Magnesium, Iron, Zinc, Manganese, Copper, Iodine, Chromium, Molybdenum, Selenium, and Cobalt. In some embodiments of the compositions described herein, the compositions comprise one mineral. In some embodiments of the compositions described herein, the compositions comprise two minerals. In some embodiments of the compositions described herein, the compositions comprise three minerals. In some embodiments of the compositions described herein, the compositions comprise four minerals. In some embodiments of the compositions described herein, the compositions comprise five minerals. In some embodiments of the compositions described herein, the compositions comprise six minerals. In some embodiments of the compositions described herein, the compositions comprise seven minerals. In some embodiments of the compositions described herein, the compositions comprise eight minerals. In some embodiments of the compositions described herein, the compositions comprise nine minerals. In some embodiments of the compositions described herein, the compositions comprise 10 minerals.

Exemplary dietary supplement nutrients include omega fatty acids, fish oil, amino acids, green tea extract and other botanical extracts.

The compositions described herein comprise mixtures in some embodiments. In some embodiments, a composition comprises a homogenous mixture of one or more active ingredients with a base substance, wherein in different embodiments, the base substance respectively comprises a solid, a liquid, or a semi-solid. For example, in some embodiments, a base substance comprising a gummy includes one or more active ingredients mixed within it (i.e. within the substance of the gummy itself). In some embodiments, a composition comprises a homogenous mixture of two or more active ingredients with one another but neither of which forms a homogenous mixture with the base substance. For example, in some embodiments, a base substance entirely encapsulates a mixture of one or more active ingredients such as, for example, a gummy containing a liquid center which contains a homogenous mixture of two or more active ingredients.

In some embodiments, a composition as described herein is sealed or covered with a coating. In some embodiments, the coating comprises a sugar-based coating. In some embodiments, the coating comprises an edible wax.

Table 1 shows the content of an exemplary embodiment of the compositions described herein which comprises the active ingredients beta-glucan, Bacillus subtilis strain DE111, and B. coagulans strain IS-2, which are mixed with a gel (gel not shown in table 1).

TABLE 1

| Composition Component | Amount |
|---|---|
| Beta-glucan (Baker's Yeast Source) | 100 mg |
| Bacillus subtilis DE111 | 2.5 billion CFU |
| B. coagulans IS-2 | 1 billion CFU |

Beta-Glucan

Beta-glucan is obtained from natural sources including yeasts, grains, and mushrooms. In some embodiments, a composition as described herein comprises beta-glucan which functions in part as a prebiotic and an in part as an immune enhancer in the individual. Beta-glucan has been found to specifically boost immunity by binding to the dectin-1 cell surface receptor, which leads to enhanced phagocytosis of pathogens by macrophages and dendritic cells.

Beta-glucan functions as a prebiotic in the compositions described herein in that one or more of the living microorganisms of the composition use the beta-glucan as a food source. Beta-glucan is a substance that is difficult for humans and animals to digest, which allows it to remain available in the GI tract as a food source to the one or more microorganisms of the compositions described herein.

In some embodiments of the compositions described herein, the composition comprises about 35-200 mg of beta-glucan. In some embodiments, the composition comprises about 35 mg of beta-glucan. In some embodiments, the composition comprises about 50 mg of beta-glucan. In some embodiments, the composition comprises about 70 mg of beta-glucan. In some embodiments, the composition comprises about 75 mg of beta-glucan. In some embodiments, the composition comprises about 100 mg of beta-glucan. In some embodiments, the composition comprises about 125 mg of beta-glucan. In some embodiments, the composition comprises about 150 mg of beta-glutan. In some embodiments, the composition comprises about 175 mg of beta-glucan. In some embodiments, the composition comprises about 200 mg of beta-glucan. In some embodiments, the composition comprises about 225 mg of beta-glucan. In some embodiments, the composition comprises about 250 mg of beta-glucan. In some embodiments, the composition comprises about 275 mg of beta-glucan. In some embodiments, the composition comprises about 300 mg of beta-glucan. In some embodiments, the composition comprises about 325 mg of beta-glucan. In some embodiments, the composition comprises about 350 mg of beta-glucan. In some embodiments, the composition comprises about 375 mg of beta-glucan. In some embodiments, the composition comprises about 400 mg of beta-glucan.

As a prebiotic, beta-glucan is an active ingredient in embodiments of the composition described herein in which beta-glucan is included.

Bacillus coagulans and Bacillus subtilis

In some embodiments of the compositions described herein, the compositions comprise the probiotic Bacillus coagulans, which is a Gram-positive rod-shaped bacterium, is catalase positive, is spore-forming, is motile, and is a facultative anaerobe. B. coagulans has been shown to be effective in improving GI function, for example, in terms of the treatment of acute diarrhea. See R. Sudha et al. Efficacy of Bacillus Coagulans Strain Unique IS-2 in the treatment of Patients with Acute Diarrhea. International Journal of Probiotics & Prebiotics, February 2012, Vol. 7 Issue 1 p33-37. B. coagulans has been shown to be effective in treating vaginosis. R. Sudha et al. Clinical study of Bacillus coagulans Unique IS-2 in Treatment of Patients with Bacterial Vaginosis. Indian Journal of Microbiology.52(3): 396-399, 2011. B. coagulans has been shown to be effective in treating hypercholesterolemia. R. Sudha et al. Effect of Supplementation of Bacillus Coagulans Unique IS-2(ATCC pat-11748) on Hypercholesterolemic Subjects: a Clinical Study. international Journal of Probiotics and Prebiotics, 6,(2):89-94, 2011. In some embodiments, the composition comprises about 1 million CFU of B. coagulans. In some embodiments, the composition comprises about 10 million CFU of B. coagulans. In some embodiments, the composition comprises about 100 million CFU of B. coagulans. In some embodiments, the composition comprises about I billion CFU of B. coagulans. In some embodiments, the composition comprises about 2 billion CFU of B. coagulans. In some embodiments, the composition comprises about 3 billion CFU of B. coagulans. In some embodiments, the composition comprises about 4 billion CFU of B. coagulans. In some embodiments, the composition comprises about 5 billion CFU of B. coagulans. In some embodiments, the composition comprises about 6 billion CFU of B. coagulans. In some embodiments, the composition comprises about 7 billion CFU of B. coagulans. In some embodiments, the composition comprises about 8 billion CFU of B. coagulans. In some embodiments, the composition comprises about 9 billion CFU of B. coagulans. In some embodiments, the composition comprises about 10 billion CPU of B. coagulans.

In some embodiments, of the compositions described herein, the compositions comprise the probiotic Bacillus subtilis, which is a Gram-positive rod-shaped bacterium, is catalase positive, is spore-forming, and is classified as an obligate aerobe although evidence exists that it can function as a facultative anaerobe. B. subtilis has been shown to be effective in improving GI function generally. See G. Labellarte et al. Tolerance and Efficacy of the Probiotic DE111 Delivered in Capsule Form. The FASEB Journal, 29(1) Supplement 924.33. April, 2015. In some embodiments, the composition comprises about 1 million CFU of B. subtilis. In some embodiments, the composition comprises about 10 million CFU of B. subtilis. In some embodiments, the composition comprises about 100 million CFU of B. subtilis. In some embodiments, the composition comprises about 1 billion CFU of B. subtilis. In some embodiments, the composition comprises about 2 billion CFU of B. subtilis. In some embodiments, the composition comprises about 3 billion CFU of B. subtilis. In some embodiments, the composition comprises about 4 billion CFU of B. subtilis. In some embodiments, the composition comprises about 5 billion CFU of B. subtilis. In some embodiments, the composition comprises about 6 billion CFU of B. subtilis. In some embodiments, the composition comprises about 7 billion CFU of B. subtilis. In some embodiments, the composition comprises about 8 billion CFU of B. subtilis. In some embodiments, the composition comprises about 9 billion CFU of B. subtilis. In some embodiments, the composition comprises about 10 billion CFU of B. subtilis.

In some embodiments of the compositions described herein, the compositions comprise B. coagulans together with B. subtilis. In some embodiments, B. coagulans together with B. subtilis are mixed together with a prebiotic within the composition. In some embodiments, B. coagulans and B. subtilis organisms within the composition are encapsulated in protective spores within the composition until the organisms are delivered to the GI tract of an individual. In these embodiments the composition is configured so that B. coagulans and B. subtilis mostly remain in a dormant state (i.e. in spore form) until and pass through the stomach so that they are protected and able to survive until reaching the lower GI tract. In this way, the dormancy state of the B. coagulans and B. subtilis organisms within the composition results in the delivery of living B. coagulans and B. subtilis organisms to the GI tract of the individual. It is these living B. coagulans and B. subtilis organisms that provide the full range of health benefits to the individual, whereas non-living organisms do not.

B. coagulans and B. subtilis when provided together, as in the compositions described herein, provide a combination of health benefits beyond the health benefits that they each provide alone. For example, B. coagulans has been shown to be an excellent colonizer of the GI tract relative to other species of probiotics. That is, B. coagulans that are ingested adhere to surfaces along the GI tract, survive, and reproduce. On its own, B. subtilis is a less effective colonizer of the intestinal tract as studies show that it typically colonizes for a relatively short duration. In the compositions described herein that provide B. coagulans together with B. subtilis, the presence of B. coagulans produces an environment that promotes improved colonization of B. subtilis. In this way, the beneficial effects provided by B. subtilis are enhanced and provided over a longer period of time (i.e. because the colonization is improved). Thus, B. subtilis in combination with B. coagulans provides an enhanced health benefit to the individual over B. subtilis alone, and when B. subtilis is combined with B. coagulans, the health benefits of B. subtilis add to the health benefits of B. coagulans.

In some embodiments of the compositions described herein, the strain of B. coagulans that is used is IS-2 and the strain of B. subtilis that is used is DE-111.

As probiotics B. subtilis and B. coagulans are active ingredient in embodiments of the composition described herein in which they are included

Manufacture

In some embodiments of the compositions described herein, the compositions are manufactured as follows: A dry base substance is added to a quantity of water and mixed forming a hydrocolloid. In an optional step, one or more sweeteners are added to the hydrocolloid and mixed at this stage. The hydrocolloid mixture is then heated to promote solidification of the hydrocolloid mixture, non-limiting examples of the solidified (or semi-solid) hydrocolloid include a gum, a gelatin, a starch, a pectin, and a gel. In some embodiments, the temperature to which the hydrocolloid is heated is about 240 degrees Fahrenheit. In some embodiments, the hydrocolloid mixture is heated by passing it through a heated chamber. The heated mixture is then cooled to a temperature below about 185 degrees Fahrenheit. In some embodiments, the hydrocolloid mixture is cooled under reduced pressure so that evaporation of fluid occurs thus further concentrating and thus further the hydrocolloid. The cooled hydrocolloid is then further mixed, and, optionally, flavoring agents, coloring agents, or texture providing agents are added at this stage. An acid, such as citric acid, is added this stage in some embodiments and acts to promote further solidification or gelling of the hydrocolloid by lowering the pH of the mixture. Active ingredients are also added at this stage and mixed into the cooled hydrocolloid. In some embodiments, the active ingredients are premixed before adding to the hydrocolloid. In some embodiments, the active ingredients added to a hydrocolloid comprising a gel include about 35-200 mg of beta-glutamine, about 1 to 3 billion colony forming units of B. coagulans in mostly spore form, and about 3 to 5 billion colony forming units of B. subtilis in mostly spore form. In some embodiments, to ensure that the proper amount of active B. coagulans and B. subtilis are delivered to the individual, double the amount that is targeted for delivery is mixed with the hydrogel during manufacture. So for example, in these embodiments, if 5 billion colony forming units are to be delivered of B. subtilis The mixture of hydrocolloid and active ingredients is then placed into a mold and undergoes a conditioning period where the hydrocolloid mixture further solidifies. In an optional finishing step, a coating is added to the hydrocolloid and active ingredient mixture. In some embodiments, the coating comprises a sugar coating and in some embodiments the coating comprises a wax coating.

Conditions Treated

Embodiments of the compositions described herein provide GI and/or immune enhancing health benefits when ingested.

As such, the compositions described herein are useful in treating and relieving the symptoms of GI infectious and inflammatory processes, such as, for example, bacterial infection, C. difficile overgrowth, inflammatory bowel disease, peptic ulcers, inflammation, colitis, necrotizing enterocolitis, and irritable bowel syndrome.

Systemic health benefits include treatment and relief of, for example, vaginosis, urogenital infection, certain cancers, high cholesterol, high blood pressure, immunity function, immune based infections, eczema.

Further, by improving bowel function generally, health benefits provided by the compositions described herein also include decreased constipation, decreased gas and bloating, decreased incidence of hemorrhoids (due to increased transit time and thus more well-formed stool), less GI discomfort, and increased regularity of bowel function.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A composition comprising:
a hydrocolloid;
a beta-glucan;
a first spore forming bacteria; and
a second spore forming bacteria;
wherein the composition comprises a mixture of the hydrocolloid, the beta-glucan, the first spore forming bacteria, and the second spore forming bacteria.

2. The composition of claim 1, wherein the hydrocolloid comprises at least one of a gum, a gelatin, a starch, a gel, and a pectin.

3. The composition of claim 1, comprising a wax coating that encapsulates the composition.

4. The composition of claim 1, wherein the hydrocolloid comprises 85% by weight or more of one or more carbohydrates.

5. The composition of claim 1, wherein the hydrocolloid comprises 1% by weight or more of an acid.

6. The composition of claim 1, wherein the beta-glucan is derived from a grain or a fungus.

7. The composition of claim 1, comprising from about 70 mg to about 200 mg of beta-glucan.

8. The composition of claim 1, wherein the first spore forming bacteria and the second spore forming bacteria are each selected from a group consisting of Bacillus subtilis, Bacillus clausii, Bacillus cereus, Bacillus coagulans and Bacillus licheniformis.

9. The composition of claim 1, wherein the first spore forming bacteria comprises about 1.5 billion to about 2.5 billion colony forming units of Bacillus subtilis and the second spore forming bacteria comprises about 0.5 billion to about 1 billion colony forming units of Bacillus coagulans.

10. The composition of claim 1, wherein the composition comprises a homogenous mixture.

11. A method of treating an individual, comprising:
providing the individual with a composition comprising a mixture of a hydrocolloid, a beta-glucan, a first spore forming bacteria, and a second spore forming bacteria.

12. The method of claim 11, wherein the hydrocolloid comprises at least one of a gum, a gelatin, a starch, a gel, and a pectin.

13. The method of claim 11, wherein the composition comprises a wax coating that encapsulates the composition.

14. The method of claim 11, wherein the hydrocolloid comprises 85% by weight or more of one or more carbohydrates.

15. The method of claim 11, wherein the hydrocolloid comprises 1% by weight or more of an acid.

16. The method of claim 11, wherein the beta-glucan is derived from a grain or a fungus.

17. The method of claim 11, wherein the composition comprises from about 35 mg to about 200 mg of beta-glucan.

18. The method of claim 11, wherein the first spore forming bacteria and the second spore forming bacteria are each selected from a group consisting of Bacillus subtilis, Bacillus clausii, Bacillus cereus, Bacillus coagulans and Bacillus licheniformis.

19. The method of claim 11, wherein the first spore forming bacteria comprises about 1.5 billion to about 2.5 billion colony forming units of Bacillus subtilis and the second spore forming bacteria comprises about 0.5 billion to about 1 billion colony forming units of Bacillus coagulans.

20. The composition of claim 11, wherein the composition comprises a homogenous mixture.
